(19) United States
(12) Patent Application Publication
(10) Pub. No.: US 2026/0131315 A1
(43) Pub. Date: May 14, 2026

(54) Ag3VO4 PHOTOCATALYST FOR CONVERSION OF CARBON DIOXIDE TO SOLAR FUELS

(71) Applicant: KING FAHD UNIVERSITY OF PETROLEUM AND MINERALS, Dhahran (SA)

(72) Inventors: Abdullah Salem Mohammed BAFAQEER, Dhahran (SA); Aniz Chennampilly UMMER, Dhahran (SA)

(73) Assignee: KING FAHD UNIVERSITY OF PETROLEUM AND MINERALS, Dhahran (SA)

(21) Appl. No.: 18/943,408

(22) Filed: Nov. 11, 2024

(51) Int. Cl. B01J 23/68; B01J 35/39; B01J 35/45; B01J 35/50; B01J 37/04; C07C 1/12

(52) U.S. Cl. CPC B01J 23/682; B01J 35/39; B01J 35/45; B01J 35/50; B01J 37/04; C07C 1/12; B01J 2235/15; B01J 2235/30; C07C 2523/68

(57) ABSTRACT

A photocatalyst that includes Ag₃VO₄ nanowires for photocatalytic reduction of carbon dioxide to solid fuels and a method for the fabrication of the photocatalyst. The Ag₃VO₄ nanowires have a hierarchical structure that includes primary nanorods having a mean diameter of 2.5 to 50 nm arranged into nanowire bundles having a mean diameter of 100 to 500 nm. The photocatalyst is used a method of photocatalytically reducing carbon dioxide to methane, for example, using solar spectrum irradiation.

### STATEMENT OF PRIOR DISCLOSURE BY AN INVENTOR

Aspects of the present disclosure are described in Abdullah Bafaqeer, Aniz Chennampilly Ummer, and Duraisami Dhamodharan; “Hierarchical Ag₃VO₄ Nanorods as an Excellent Visible Light Photocatalyst for CO₂ Conversion to Solar Fuels”; Catalysts, 2024, 14(10), 672, which is incorporated herein by reference in its entirety.

### BACKGROUND

### Technical Field

The present disclosure is directed to a photocatalyst that includes Ag₃VO₄ nanoparticles that is used in a method of the photocatalytic conversion of carbon dioxide to methane as solar fuels.

### Description of the Background

The “background” description provided herein is to present the context of the disclosure generally. Work of the presently named inventors, to the extent it is described in this background section, as well as aspects of the description that may not otherwise qualify as prior art at the time of filing, are neither expressly nor impliedly admitted as prior art against the present invention.

Solar energy is most abundant in the world. However, it is difficult to store the produced electric energy in large quantities using the present technologies. A solar energy-based technology to recycle carbon dioxide into readily transportable hydrocarbon fuel (i.e., a solar fuel) would help reduce atmospheric CO₂ levels and partly fulfill energy demands within the present hydrocarbon-based fuel infrastructure. The photocatalytic production of chemical fuels from CO₂ using sunlight (solar fuels) is a promising strategy for addressing global environmental problems and securing future energy supplies. The potential of photocatalytic CO₂ conversion is significant for the production of fuels and chemicals without the need for tapping sequestered carbons in the form of fossil fuels, while simultaneously mitigating CO₂ emissions and addressing environmental concerns.

Although extensive research has been conducted to date, numerous obstacles to realizing efficient, selective, and stable CO₂ reduction have yet to be overcome. The past several decades have seen a significant rise in atmospheric carbon dioxide levels resulting from the combustion of hydrocarbon fuels. A current technology promising for CO₂ reduction is CO₂ capture and storage (CCS), which merely stores carbon dioxide as a gas, liquid, or solid. An alternative technology is to capture CO₂ and convert it chemically by thermal catalysis. However, this technology suffers from certain drawbacks and limitations that prevent its widespread adoption. For example, is not appropriate to low concentration of CO₂ (<1%), such as CO₂ in air. Further, conventional thermal catalysts must be driven by thermal energy, mainly produced by fossil fuel combustion. The solar energy driven CO₂ conversion is advantageous because it does not contribute extra CO₂ emission (carbon neutral process). There is still a pressing need to exploit non-thermal methods to easily convert and store solar energy alongside discovering new technologies to largely store electric energy.

In recent years, there has been significant interest in the solar photocatalytic CO₂ reduction into high-value fuels. Currently, single metal-based photocatalysts are used for solar photocatalytic CO₂ reduction, but these photocatalyst suffer from drawbacks such as low performance, uncontrollable selectivity, and instability. Numerous research efforts have been dedicated to developing efficient photocatalysts for CO₂ reduction, including V₂O₅[H. Fu, et. al., Appl. Surf. Sci., 622 (2023) 156964], TiO₂ [Z. Li, et. al., Sep. Purif. Technol., 330 (2024) 125250.; A. Bafaqeer, et. al., Chem. Eng. Res. Des., 201 (2024) 353-361; and X. Guo, et. al., J. Environ. Chem. Eng., 12 (2024) 111990], Ta₂O₅ [X. Shao, et. al., Chinese J. Catal., 51 (2023) 193-203], ZnO [J. Li, et. al., Nano Materials Science, 5 (2023) 237-245; and H. A. Thabit, et. al., Optical Materials, 137 (2023) 113548], WO₃ [M. Chico-Vecino, et. al., J. Environ. Chem. Eng., 11 (2023) 110372], and CdS [M. Zhao, et. al., Sep. Purif. Technol., 329 (2024) 125179]. Notably, V₂O₅ has been extensively studied as a semiconductor catalyst due to its potent oxidation and reduction capabilities. Furthermore, it is environmentally friendly, cost-effective, and can be synthesized in various nanostructures through economical methods. [R. Espinosa, et. al., Environ. Sci. Pollut. Res., 30 (2023) 54119-54129]. However, its high charge recombination rate has led to subpar photocatalytic performance. Among many candidate materials, α-Fe₂O₃ has been extensively investigated to develop highly active photocatalysts. Despite its suitable energy band position, α-Fe₂O₃ suffers from the relative low reduction capacity and charge separation ability, which are detrimental to the photocatalytic efficiency.

Photocatalytic carbon dioxide (CO₂) conversion into solar fuels is an effective means for simultaneously solving both the greenhouse effect and the energy crisis. In the past ten years, bismuth (Bi)-based photocatalysts for environmental remediation have experienced a golden development period. In the past ten years, Bi-based photocatalysts have been suggested as excellent visible light photocatalytic materials due to their suitable bandgap. However, energy applications of photocatalysis, such as water splitting, N₂ fixation, and CO₂ conversion over pristine Bi-based photocatalysts are limited due to low reduction abilities and low electron-hole separation efficiencies. For applications in CO₂ conversion, an elevated conduction band (CB) position is required for Bi-based photocatalysts. In addition, to promote electron migration to the photocatalyst surface and enable electron participation in reduction reactions by improving the charge separation efficiency, co-catalysts, surface modification, or heterojunctions are used.

Accordingly, it is one object of the present disclosure to develop a highly efficient visible light photocatalyst for the conversion of carbon dioxide (CO₂) into solar fuels which indirectly serves as a great potential in harnessing solar energy to drive the conversion of CO₂, a greenhouse gas, into valuable solar fuels which does not suffer from the above-identified drawbacks.

### SUMMARY

According to a first aspect, the present application relates to a photocatalyst. In some embodiments, the photocatalyst includes Ag₃VO₄ nanowires. In some embodiments, the catalytic Ag₃VO₄ nanowires have a hierarchical structure and includes primary nanorods having a mean diameter of 2.5 to 50 nanometer (nm) arranged into nanowire bundles having a mean diameter of 100 to 500 nm.

In some embodiments, the photocatalyst the catalytic Ag₃VO₄ nanowires are crystalline monoclinic Ag₃VO₄ by Powder X-ray Diffraction (PXRD).

In some embodiments, the catalytic Ag₃VO₄ nanowires have a mean primary nanorod length of 1 to 15 micrometers (μm).

In some embodiments, the photocatalyst is substantially free of V₂O₅, Ag₃O, and Ag₂O by PXRD.

In some embodiments, the photocatalyst has a band gap of 2.05 to 2.35 electron volt (eV).

In some embodiments, the photocatalyst has a valence band energy of 1.71 to 2.01 eV; and a conduction band energy of −0.64 to −0.04 eV.

In some embodiments, the photocatalyst further includes a substrate on which the catalytic Ag₃VO₄ nanowires are disposed.

In some embodiments, the substrate is at least one selected from the group consisting of glass, quartz, indium tin oxide, fluorine tin oxide, and aluminum zinc oxide.

The present disclosure also relates to a method of forming the photocatalyst. In some embodiments, the method includes mixing a first solution including a silver source in water with a second solution including a metavanadate salt in water to form a first reaction mixture. In some embodiments, the method further includes aging the reaction mixture for 1 to 48 hours while stirring to form a first product. In some embodiments, the method further includes washing the first reaction product with a distilled water to form a washed product which is then followed by drying the washed product at 60 to 100° C. for 1 to 24 hours to form the catalytic Ag₃VO₄ nanowires.

In some embodiments, the silver source is silver nitrate.

In some embodiments, the metavanadate salt is ammonium metavanadate.

In some embodiments, the method further includes disposing the catalytic Ag₃VO₄ nanowires on a substrate.

In some embodiments, the substrate is at least one selected from the group consisting of glass, quartz, indium tin oxide, fluorine tin oxide, and aluminum zinc oxide.

The present disclosure also relates to a method of reducing carbon dioxide to methane. In some embodiments, the method includes contacting the photocatalyst with a gaseous mixture including carbon dioxide and water to form a reduction mixture, irradiating the reduction mixture with visible light to form a product mixture including methane, and collecting the product mixture.

In some embodiments, the irradiating of the reduction mixture is performed with a visible light intensity of 50 watt (W).

In some embodiments, the gaseous mixture is contacted with the photocatalyst at a rate of 5 to 15 milliliter per minute (mL/min).

In some embodiments, the method produces 120 to 200 μmol methane per g of photocatalyst after 1 hour of irradiation.

In some embodiments, the gaseous mixture is substantially free of oxygen.

In some embodiments, the product mixture further includes dimethyl ether.

In some embodiments, the product mixture has a ratio of methane to dimethyl ether of 1:1 to 8:1.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A shows a schematic flow chart of a method of forming a photocatalyst, according to certain embodiments.

FIG. 1B shows a schematic flow chart of a method of reducing carbon dioxide to methane, according to certain embodiments.

FIG. 2 is a schematic diagram of a formation process of Ag₃VO₄ nanorods, according to certain embodiments.

FIG. 3 is a schematic diagram of an experimental setup for photocatalytic CO₂ conversion with H₂O, according to certain embodiments.

FIG. 4 shows a X-ray diffraction (XRD) pattern of the Ag₃VO₄ nanorods, according to certain embodiments.

FIG. 5A shows a scanning electron microscope (SEM) image of Ag₃VO₄ nanorods prepared at room temperature after 6 h stirring, according to certain embodiments.

FIG. 5B shows a SEM image of Ag₃VO₄ nanorods prepared at room temperature after 12 h stirring, according to certain embodiments.

FIG. 5C shows a SEM image of Ag₃VO₄ nanorods prepared at room temperature after 24 h stirring, according to certain embodiments.

FIG. 5D shows the morphologies of Ag₃VO₄ photocatalyst prepared at room temperature for different stirring times through energy dispersive X-ray analysis (EDX) spectrum, according to certain embodiments.

FIG. 5E shows the elemental composition of Ag₃VO₄ photocatalyst prepared at room temperature for different stirring times through energy dispersive X-ray analysis (EDX) spectrum, according to certain embodiments.

FIG. 5F shows a transmission electron microscopy (TEM) image of Ag₃VO₄ photocatalyst prepared at room temperature after 2.4 h stirring, according to certain embodiments.

FIG. 5G shows a high-resolution transmission electron microscopy (HR-TEM) image of lattice fringes of Ag₃VO₄ photocatalyst, according to certain embodiments.

FIG. 5H shows a selected area electron diffraction (SAED) pattern of Ag₃VO₄ photocatalyst, according to certain embodiments.

FIG. 6A shows a diffuse reflectance (DR) UV-vis spectra of Ag₃VO₄ photocatalyst, according to certain embodiments.

FIG. 6B shows a Tauc plot for band gap energy calculations from absorption spectra of Ag₃VO₄ photocatalyst, according to certain embodiments.

FIG. 7 is a bar graph depicting yield of methane (CH₄) and di-methyl ether (DME) over various photocatalysts (irradiation time=1 h, room temperature, and atmospheric pressure), according to certain embodiments.

FIG. 8 shows a bar graph of hydrocarbon yield for different reaction times of stirring for 6 h, 12 h and 24 h during the preparation of Ag₃VO₄ sample for carbon-dioxide (CO₂) photoconversion (irradiation time=1 h, room temperature, and atmospheric pressure), according to certain embodiments.

FIG. 9 shows a plot of the hydrocarbon yield vs irradiation time, showing the effect of irradiation time on the performance of Ag₃VO₄ for the production of methane (CH₄) and dimethyl ether (DME), according to certain embodiments.

FIG. 10 is a schematic diagram of a reaction mechanism proposed for the photo reduction of CO₂ to CH₄ and DME over Ag₃VO₄ nanorods under visible light, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Embodiments of the present disclosure will now be described more fully hereinafter with reference to the accompanying drawings, in which some, but not all embodiments of the disclosure are shown.

As used herein, the words “about,” “approximately,” or “substantially similar” may be used when describing magnitude and or position to indicate that the value and/or position described is within a reasonable expected range of values and/or positions. For example, a numeric value may have a value that is +/−0.1% of the stated value (or range of values), +/−1% of the slated value (or range of values), +/−2% of the stated value (or range of values), +/−5% of the slated value (or range of values), +/−10% of the staled value (or range of values), +/−15% of the stated value (or range of values), or +/−20% of the stated value (or range of values). Within the description of this disclosure, where a numerical limit or range is stated, the endpoints are included unless stated otherwise. Also, all values and subranges within a numerical limit or range are specifically included as if explicitly written out.

As used herein, the term ‘photocatalyst’ refers to a substance that accelerates a chemical reaction through the absorption of light. Specifically, a photocatalyst absorbs photons and utilizes the energy to drive redox reactions, often facilitating the conversion of environmental pollutants or greenhouse gases into valuable products. In the context of this invention, a photocatalyst is a material that enhances the conversion of carbon dioxide (CO₂) into solar fuels by harnessing visible light, thus playing a critical role in photocatalytic processes aimed at sustainable energy and environmental remediation.

As used herein, the term ‘chemical deposition method’ refers to a technique for creating thin films or nanostructures by depositing a chemical substance onto a substrate. This process involves the use of chemical reactions to form a solid layer or structure from gaseous or liquid precursors. The chemical deposition method encompasses various approaches, such as chemical vapor deposition (CVD), solution-based deposition, and electrochemical deposition, each of which utilizes different principles and conditions to achieve the desired material properties. In the context of this invention, the chemical deposition method is specifically employed to synthesize Ag₃VO₄ nanorods, where chemical precursors are combined to form and deposit the nanorods onto a substrate or within a solution, resulting in the formation of the desired photocatalytic material.

Aspects of this disclosure are directed to a method of synthesis of hierarchical Ag₃VO₄ nanorods, engineered to function as a highly efficient visible light photocatalyst for the conversion of carbon dioxide (CO₂) into solar fuels. The photocatalyst of the present disclosure demonstrates remarkable potential in harnessing solar energy to drive the conversion of CO₂, a greenhouse gas, into valuable solar fuels, contributing to the mitigation of climate change and the pursuit of sustainable energy solutions.

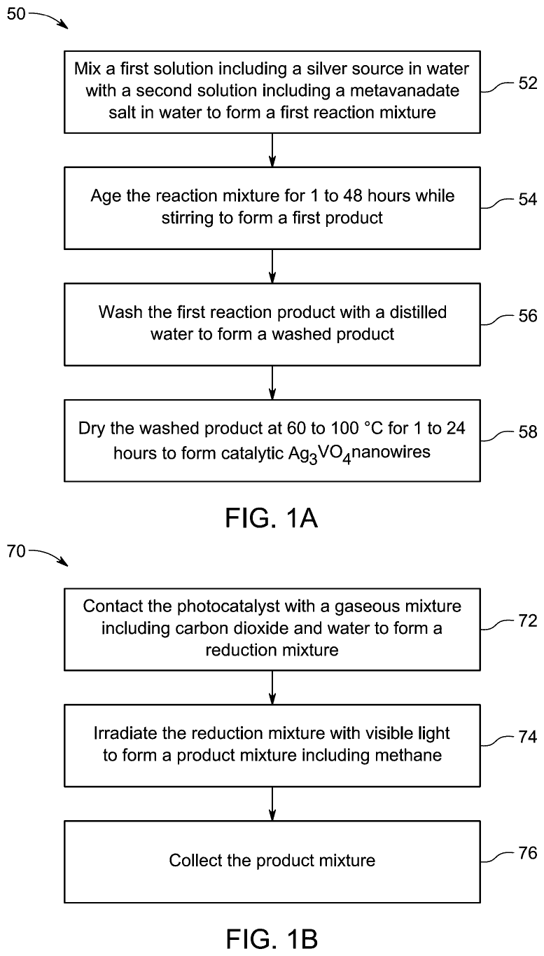

FIG. 1A illustrates a schematic flow chart of a method 50 of synthesis of Ag₃VO₄ nanorods using a chemical deposition method according to certain embodiments of the present disclosure. The order in which the method 50 is described is not intended to be construed as a limitation, and any number of the described method steps can be combined to implement the method 50. Additionally, individual steps may be removed or skipped from the method 50 without departing from the spirit and scope of the present disclosure.

At step 52, the method 50 includes mixing a first solution including a silver source in water with a second solution including a metavanadate salt in water to form a first reaction mixture. In some embodiments, second solution may have a metavanadate concentration of 0.05 to 2.0 M, preferably 0.1 to 1.5 M, preferably 0.15 to 1.0 M, preferably 0.25 to 0.75 M, preferably 0.3 to 0.7 M, preferably 0.35 to 0.65 M, preferably 0.40 to 0.60 M, preferably 0.45 to 0.55 M, preferably 0.49 to 0.51 M, preferably about 0.5 M. In some embodiments, the first solution may have a silver ion concentration of 0.05 to 2.0 M, preferably 0.1 to 1.5 M, preferably 0.15 to 1.0 M, preferably 0.25 to 0.75 M, preferably 0.3 to 0.7 M, preferably 0.35 to 0.65 M, preferably 0.40 to 0.60 M, preferably 0.45 to 0.55 M, preferably 0.49 to 0.51 M, preferably about 0.5 M. In some embodiments, the reaction mixture, once formed, may be stirred. In some embodiments, the stirring may be performed for 5 to 90 minutes, preferably 15 to 75 minutes, preferably 30 to 60 minutes, preferably 45 minutes. Stirring may be advantageous for ensuring that the mixture remains homogeneous and that the reactants are evenly distributed, which may allow for consistent and efficient product formation.

In general, the silver source can be any suitable source of silver ions (e.g., Ag⁺). Examples of suitable silver sources include, but are not limited to, silver nitrate, silver chloride, silver acetate, silver oxide, silver phosphate, silver iodide, silver bromide, silver oxalate, and/or hydrates thereof. In some embodiments, the silver source is silver nitrate.

In some embodiments, the metavanadate salt may include, but is not limited to, sodium metavanadate salt, potassium metavanadate salt, ammonium metavanadate salt, calcium metavanadate, magnesium metavanadate, and/or hydrates thereof. In some embodiments, the metavanadate salt is ammonium metavanadate salt.

At step 54, the method 50 includes aging the reaction mixture for 1 to 48 hours, preferably 2 to 46 hours, preferably 4 to 44 hours, preferably 6 to 42 hours, preferably 8 to 40 hours, preferably 10 to 38 hours, preferably 12 to 36 hours, preferably 14 to 34 hours, preferably 16 to 32 hours, preferably 18 to 30 hours, preferably 20 to 28 hours, preferably 22 to 26 hours, preferably 24 hours while stirring to form a first product. During this time, the reaction mixture, which includes the silver source and metavanadate salt in solution, undergoes a series of chemical and physical changes. The aging may be advantageous for ensuring that the reaction reaches completion, or that the resulting product has the desired properties for subsequent processing or application.

At step 56, the method 50 includes washing the first reaction product to form a washed product. The washing step may be advantageous for purifying the solid material formed during the reaction by removing any residual reactants, by-products, or unreacted chemicals that may be present. In some embodiments, the washing is performed with water. In some embodiments, the washing is performed with distilled water. The use of distilled water may be advantageous for ensuring that the washing process does not introduce any additional impurities or contaminants that could interfere with the quality or functionality of the final product and/or for ensuring removal of any residual reactants, by-products, or unreacted chemicals that may be present. In general, the washing can be performed using any suitable technique and with any suitable equipment. In some embodiments, the washing process involves immersing the reaction product in distilled water and then subjecting it to agitation or stirring to facilitate thorough cleaning. In some embodiments, the washing may be repeated multiple times to ensure complete removal of impurities. This purification process may be advantageous for ensuring that the final material has the desired properties and performance characteristics, especially for applications such as photocatalytic reduction where purity can significantly impact effectiveness.

At step 58, the method 50 includes drying the washed product at 60 to 100° C. for 1 to 24 hours to form the catalytic Ag₃VO₄ nanowires. The drying process may be advantageous for removing any remaining moisture from the washed product, which could otherwise affect the structural integrity and catalytic properties of the final nanowires. The drying method, involving applying controlled heat, may ensures that the product is uniformly dried, and may be advantageous for preventing the formation of clumps or uneven drying that could compromise the quality of the nanowires. The temperature range of 60 to 100° C. may be advantageous as a balance between effective moisture removal and preservation of the nanowires' structural and chemical characteristics. In some embodiments, the washed product may be dried at a temperature of 60 to 100° C., preferably 65 to 95° C., preferably 70 to 90° C., preferably 75 to 85° C., preferably 80° C. Extended drying times, up to 24 hours in some embodiments, provide adequate time for thorough evaporation of solvents and water, ensuring that the final product is completely dry. Proper drying may also assist in achieving crystallinity and morphology of the Ag₃VO₄ nanowires, which are critical factors influencing their catalytic performance.

In some embodiments, the photocatalyst Ag₃VO₄ nanowires are crystalline by powder X-ray diffraction (PXRD) analysis. In general, the Ag₃VO₄ nanowires may exhibit different phases, including amorphous, orthorhombic, tetragonal, cubic structures, or combinations of these by PXRD. In some embodiments, the Ag₃VO₄ nanowires are crystalline and exhibit a monoclinic structure by PXRD. Such Ag₃VO₄ nanowires showing a crystalline monoclinic structure may be referred to as “monoclinic Ag₃VO₄ nanowires” or similar term. The Ag₃VO₄ nanowires exhibiting a crystalline monoclinic structure by PXRD can indicate that the Ag₃VO₄ nanowires include monoclinic Ag₃VO₄. The monoclinic Ag₃VO₄ nanowires can also include amorphous material, such as amorphous Ag₃VO₄ and/or other crystalline phases, such as non-monoclinic crystalline Ag₃VO₄ or a crystalline material other than Ag₃VO₄. In some embodiments, the monoclinic Ag₃VO₄ nanowires are substantially free of material other than Ag₃VO₄. The characteristic diffraction patterns of Ag₃VO₄ nanowires shows notable peaks at 2θ values of 19.6°, 21.6°, 31.7°, 32.6°, 35.3°, 38.9°, 41.5°, 43.3°, 46.1°, 53.7°, and 54.05°, corresponding to the (011), (−111), (−121), (121), (301), (022), (320), (400), (−213), and (132) planes of its monoclinic crystal structure. The monoclinic structure may be particularly advantageous due to its favourable photocatalytic properties. Further, the monoclinic structure may provide a favourable bandgap and electronic configuration that enhances light absorption and improves charge separation efficiency.

In some embodiments, the photocatalyst is predominantly composed of Ag₃VO₄ by PXRD.

In some embodiments, the photocatalyst is substantially free from contaminants such as V₂O₅, Ag₃O, and Ag₂O by PXRD. The absence of characteristic peaks for V₂O₅, Ag₃O, and Ag₂O further confirms the purity of the Ag₃VO₄ phase. Specifically, V₂O₅ would exhibit peaks at approximately 20.6°, 26.2°, 35.7°, 43.8°, and 49.4° 2θ, corresponding to its orthorhombic structure; Ag₃O would show peaks around 22.7°, 30.8°, and 35.6° 2θ, indicative of its cubic or near-cubic structure; and Ag₂O would present peaks at 29.6°, 32.0°, 46.2°, and 54.8° 2θ, reflecting its cubic crystal structure.

In some embodiments, the exclusion of these unwanted phases is advantageous for ensuring that the photocatalyst retains its optimal photocatalytic performance. Contaminants such as V₂O₅, Ag₃O, and Ag₂O could potentially alter the electronic properties and crystalline integrity of the Ag₃VO₄, thereby diminishing its efficacy.

In some embodiments, the Ag₃VO₄ nanowires possess a hierarchical structure. In some embodiments, the hierarchical structure includes primary nanorods arranged to form bundles. In some embodiments, the Ag₃VO₄ material includes nanorods organized into bundles to form nanowires. In some embodiments, the primary nanorods have an average diameter of 1 to 80 nm, preferably 2 to 60 nm, preferably 2.5 to 50 nm. In some embodiments, the primary nanorods have an average length of 0.5 to 30 μm, preferably 1 to 20 μm, preferably 1.5 to 15 μm. In some embodiments the nanowires (e.g., the bundles of primary nanorods) have a mean diameter of 100 and 1000 nm, preferably 200 to 750 nm, preferably 300 to 500 nm. In some embodiments, the nanowires (e.g., the bundles of primary nanorods) have an average length of 0.5 to 30 μm, preferably 1 to 20 μm, preferably 1.5 to 15 μm. In general, the primary nanorods can be arranged into bundles in any suitable manner or arrangement. In some embodiments, the primary nanorods are arranged to have a common center or midpoint. Such an arrangement can, if the primary nanorods have different lengths, cause the nanowires to have a tiered or terraced end with different primary nanorods ending at different lengths from the midpoint. In some embodiments, the primary nanorods are arranged to have a common endpoint. In such an embodiment, the nanowires can have one or both ends substantially flattened as all the primary nanorods have a common endpoint. In some embodiments, the primary nanorods are arranged substantially parallel to one another. In some embodiments, the primary nanorods are connected to one another. Such connection between the primary nanorods can be formed by a material such as an amorphous Ag₃VO₄ or material other than Ag₃VO₄. Such connection between primary nanorods can be formed by the crystalline Ag₃VO₄ primary nanorods themselves. That is, in some embodiments, the Ag₃VO₄ nanowires are a composite monolith type structure formed from multiple primary nanorods connected and forming different domains within the monolith. This specific structural configuration may contribute to the enhanced performance and functionality of the Ag₃VO₄ nanowires in their photocatalytic applications.

In some embodiments, the photocatalyst is capable of absorbing light in the visible region. In some embodiments, the photocatalyst has a peak absorbance at a wavelength 400 to 700 nm, preferably from 450 to 650 nm, preferably 500 to 600 nm, preferably 525 to 575 nm, preferably 550 to 570 nm, preferably 563 nm. In some embodiments, the photocatalyst has a band gap of 2.05 to 2.35 eV, preferably 2.10 to 2.30 eV, preferably 2.15 to 2.25 eV, preferably 2.17 to 2.22 eV, preferably 2.2 eV. The energy band gap (E_{g}) of the Ag₃VO₄ photocatalyst may be determined using the Tauc equation, a typical method for evaluating the optical band gap of semiconductors from UV-visible absorption spectra. The Tauc equation is depicted in Equation 1

$${E}_{g} ( eV ) = \frac{1240}{λ} \qquad ( 1 )$$

In some embodiments, the photocatalyst has a valence band energy of 1.71 to 2.01 eV, preferably 1.73 to 1.99 eV, preferably 1.75 to 1.97 eV, preferably 1.77 to 1.95 eV, preferably 1.79 to 1.93 eV, preferably 1.81 to 1.91 eV, preferably 1.83 to 1.89 eV, preferably 1.85 to 1.87 eV, preferably 1.86 eV. embodiments, the photocatalyst has a conduction band energy of −0.64 to −0.04 eV, preferably −0.60 to −0.08 eV, preferably −0.56 to −0.12 eV, preferably −0.52 to −0.16 eV, preferably −0.48 to −0.20 eV, preferably −0.44 to −0.24 eV, preferably −0.42 to −0.26 eV, preferably −0.40 to −0.28 eV, preferably −0.38 to −0.30 eV, preferably −0.36 to −0.32 eV, preferably −0.34 eV.

The specific valence band energy and/or conduction band energy of the Ag₃VO₄ nanowire catalyst may be advantageous for providing predominantly CH₄ as the main product. Such a predominance of methane as the product may be due to the suitable reduction potential of CO₂/CH₄ (−0.24 V). The reaction is more favorable for CH₄ production as the reduction potential of CO₂/CH₄ (−0.24 V) is lower than the conduction band of Ag₃VO₄.

In general, the valence band energy and the conduction band energy can be determined by any suitable technique known to one of ordinary skill in the art. The conduction band position (E_{CB}) of the Ag₃VO₄ nanowires may adhere to the relationship defined using Equation (2).

$${E}_{VB} = {E}_{CB} + {E}_{bg} \qquad ( 2 )$$

In some embodiments, Ag₃VO₄ nanowires are disposed on a substrate. In some embodiments, the substrate is at least one selected from glass, quartz, indium tin oxide, fluorine tin oxide, and aluminum zinc oxide. In some embodiments, the Ag₃VO₄ nanowires are disposed on the substrate such that a length of the nanowires is substantially perpendicular to a surface of the substrate. That is, the Ag₃VO₄ nanowires are standing on end. In some embodiments, the Ag₃VO₄ nanowires are disposed on the substrate such that a length of the nanowires is substantially parallel to a surface of the substrate. That is, the Ag₃VO₄ nanowires are laying on the substrate. In some embodiments, the Ag₃VO₄ nanowires are disposed on the substrate such that a length of the nanowires forms an acute angle with respect a surface of the substrate. That is, the Ag₃VO₄ nanowires are standing at an angle with respect to the substrate. In some embodiments, the Ag₃VO₄ nanowires are disposed in a uniform orientation on the substrate. That is, all or substantially all of the Ag₃VO₄ nanowires are similarly oriented. For example, if the Ag₃VO₄ nanowires are standing on end, all or substantially all of the Ag₃VO₄ nanowires are standing on end. If the Ag₃VO₄ nanowires are angled, all or substantially all of the Ag₃VO₄ nanowires are angled. In embodiments where the Ag₃VO₄ nanowires are angled, the angle and/or direction of the nanowires may be uniform. In some embodiments, the Ag₃VO₄ nanowires are not disposed in a uniform orientation on the substrate. In some embodiments, the Ag₃VO₄ nanowires are disposed with a uniform spacing on the substrate. In some embodiments, the Ag₃VO₄ nanowires are not disposed with a uniform spacing on the substrate.

FIG. 1B illustrates an exemplary schematic flow chart of a method 70 for the reduction of carbon dioxide to methane according to certain embodiments of the present disclosure. The order in which the method 70 is described is not intended to be construed as a limitation, and any number of the described method steps can be combined to implement the method 70. Additionally, individual steps may be removed or skipped from the method 70 without departing from the spirit and scope of the present disclosure.

At step 72, the exemplary method 70 includes contacting the photocatalyst Ag₃VO₄ with a gaseous mixture including carbon dioxide and water to form a reduction mixture. In some embodiments, the gaseous mixture is contacted with the photocatalyst at a rate of 5 to 15 mL/min, preferably 6 to 14 mL/min, preferably 7 to 13 mL/min, preferably 8 to 12 mL/min, preferably 9 to 11 mL/min, preferably 10 mL/min.

In some embodiments, the gaseous mixture is kept substantially free of oxygen. In some embodiments, the gaseous mixture is kept substantially free of oxygen by creating an inert atmosphere. Oxygen may interfere with the photocatalytic reaction, for example, by participating in competing oxidative processes that may consume the available reactive species and hinder the conversion of carbon dioxide (CO₂) into desired products like methane (CH₄) and dimethyl ether (DME). Additionally, the presence of oxygen may lead to the formation of unwanted oxidation byproducts and potentially deactivate the photocatalyst by capturing electrons or holes, which are crucial for the reduction of CO₂. By maintaining an oxygen-free or nearly oxygen-free environment, the photocatalyst, such as Ag₃VO₄, may be able to operate more efficiently, focusing its activity on reducing CO₂ without the detrimental effects of oxidative interference. The exclusion of oxygen may be associated with an improvement in yields of the target products and enhancement of the overall effectiveness of the photocatalytic process. In some embodiments, the inert atmosphere may be generated by introducing inert gases such as argon, helium, or neon. In some embodiments, the reactor undergoes nitrogen purging to remove other gases and establish the inert environment.

At step 74, the exemplary method 70 involves irradiating the reduction mixture with visible light. This irradiation may be useful to facilitate the photocatalytic conversion of carbon dioxide (CO₂) into methane (CH₄) and potentially other products, such as dimethyl ether (DME). In some embodiments, this irradiation is conducted using a visible light intensity of 25 to 75 W, preferably 30 to 70 W, preferably 35 to 65 W, preferably 40 to 60 W, preferably 45 to 55 W, preferably 50 W. During step 74, the reduction mixture, which includes the photocatalyst such as Ag₃VO₄ nanorods, is exposed to visible light. The outcome of this irradiation step is a product mixture that primarily contains methane, along with potentially other hydrocarbons or byproducts, depending on the specific reaction conditions and duration of the exposure.

At step 76, the method 70 includes collecting the product mixture. In some embodiments, this product mixture is composed primarily of methane (CH₄) and dimethyl ether (DME). In some embodiments, the product mixture has a ratio of methane to dimethyl ether in the range of 1:1 to 8:1, preferably 1.5:1 to 7.5:1, preferably 2:1 to 7:1, preferably 2.5:1 to 6.5:1, preferably 3:1 to 6:1, preferably 3.25:1 to 5.75:1, preferably 3.5:1 to 5.5:1, preferably 3.75:1 to 5.25:1, preferably 4:1 to 5:1, preferably 4.25:1 to 4.75:1, preferably 4.5:1.

In some embodiments, the methane production after one hour of visible light irradiation is in a range of from 150 to 200 μmol per gram of photocatalyst, preferably 155 to 190 μmol per gram of photocatalyst, preferably 160 to 180 μmol per gram of photocatalyst, preferably 165 to 175 μmol per gram of photocatalyst. In some embodiments, the dimethyl ether production after one hour of visible light irradiation is in a range of from 5 to 50 μmol per gram of photocatalyst, preferably 10 to 45 μmol per gram of photocatalyst, preferably 15 to 40 μmol per gram of photocatalyst, preferably 20 to 35 μmol per gram of photocatalyst, preferably 25 to 30 μmol per gram of photocatalyst. In some embodiments, the methane production after four hours of visible light irradiation is in a range of from 225 to 325 μmol per gram of photocatalyst, preferably 230 to 320 μmol per gram of photocatalyst, preferably 235 to 315 μmol per gram of photocatalyst, preferably 240 to 310 μmol per gram of photocatalyst, preferably 245 to 305 μmol per gram of photocatalyst, preferably 250 to 300 μmol per gram of photocatalyst, preferably 255 to 295 μmol per gram of photocatalyst, preferably 260 to 290 μmol per gram of photocatalyst, preferably 265 to 285 μmol per gram of photocatalyst, preferably 270 to 280 μmol per gram of photocatalyst. In some embodiments, the dimethyl ether production after four hours of visible light irradiation is in a range of from 10 to 100 μmol per gram of photocatalyst, preferably 15 to 95 μmol per gram of photocatalyst, preferably 20 to 90 μmol per gram of photocatalyst, preferably 25 to 85 μmol per gram of photocatalyst, preferably 30 to 80 μmol per gram of photocatalyst, preferably 35 to 75 μmol per gram of photocatalyst, preferably 40 to 70 μmol per gram of photocatalyst, preferably 45 to 65 μmol per gram of photocatalyst, preferably 50 to 60 μmol per gram of photocatalyst.

### EXAMPLES

The following examples demonstrate exemplary Ag₃VO₄ nanowires photocatalyst, a method of preparing the photocatalyst, and a method of producing methane from CO₂ using the photocatalyst as described herein. The examples are provided solely for illustration and are not to be construed as limitations of the present disclosure, as many variations are possible without departing from the spirit and scope of the present disclosure.

### Example 1: Synthesis of Ag₃VO₄Nanorods

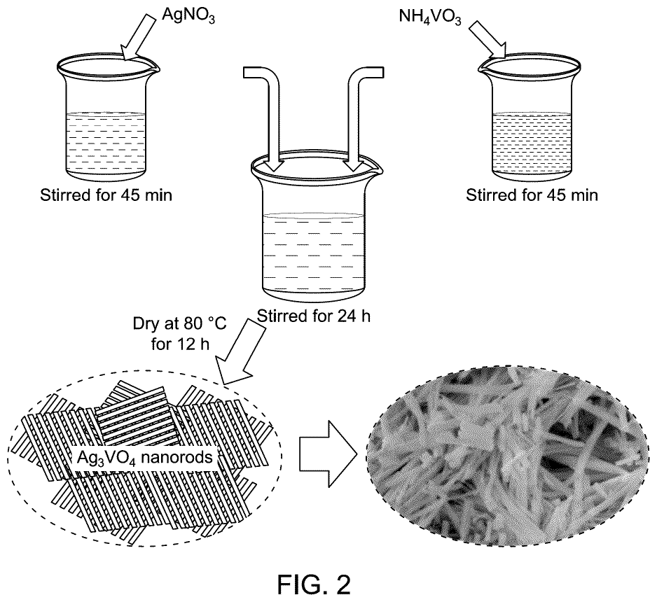

Ag₃VO₄ nanorods was prepared through a chemical deposition method. Typically, a certain amount AgNO₃ was added to 40 mL distilled water and stirred for 45 min to form solution A. Simultaneously, 2.3 g NH₄VO₃ was dissolved in 40 mL distilled water and stirred for 45 min to form a solution B. Then, the solution B was transferred to the solution A and keep stirring for 24 h. Finally, the obtained products were washed with distilled water and then dried overnight at 80° C. for 24 hr. Pristine Ag₃VO₄ nanorods were obtained. The Ag₃VO₄ samples were synthesized with varying stirring durations of 6 hours, 12 hours, and 24 hours using the same procedures. The schematic for fabricating the Ag₃VO₄ nanorods is illustrated in FIG. 2.

### Example 2: Photocatalytic Activity Test

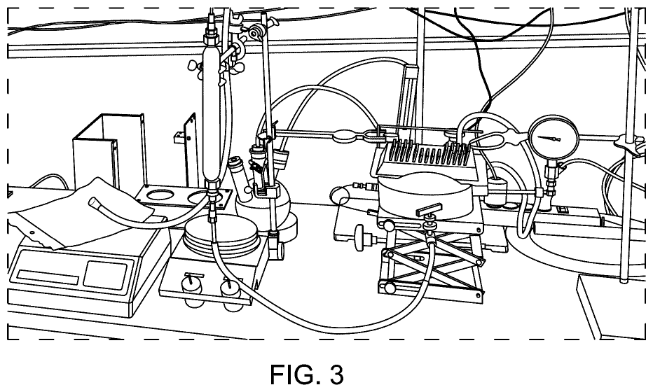

The as-prepared photocatalysts were tested for photocatalytic conversion of CO₂ in a gaseous phase fixed-bed photoreactor, as shown in FIG. 3. A 50 W LED flood light, coupled with a concentrator, served as the illumination source to activate the photocatalytic reactions, facilitated by a quartz window to optimize light involvement. 150 mg of powder photocatalysts were dispersed at the bottom of the photoreactor to ensure proper distribution. Prior to introducing reactant gases, the reactor underwent nitrogen purging to eliminate other gases. High-purity compressed CO₂, regulated by a mass flow controller, was passed through a water saturator for moisture carriage. For continuous CO₂ reduction, gas flowed through the reactor at a rate of 10 20 mL/min. Product analysis employed an online gas chromatograph (GC) with flame ionization detector (FID) and thermal conductivity detector (TCD) detectors (GC/FID/TCD). In the absence of light irradiation or catalysts, carbon-containing products were not detected.

### Example 3: Physical Characterization

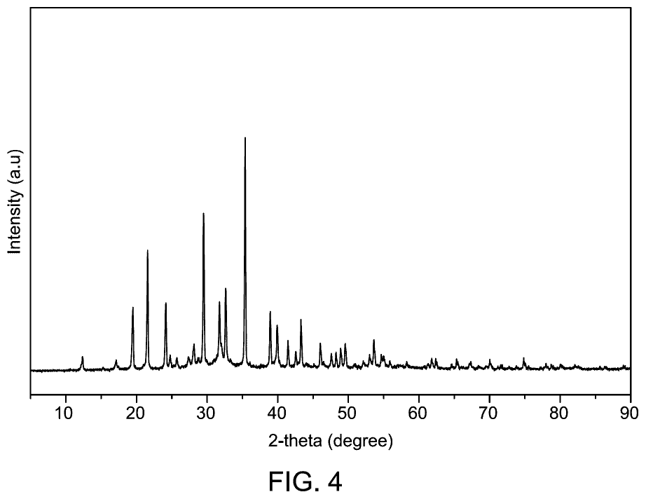

The X-ray diffraction (XRD) analysis was employed to investigate the crystal phase and structure of photocatalysts. The XRD pattern of Ag₃VO₄ photocatalyst are depicted in FIG. 4. The pure Ag₃VO₄ displayed distinct peaks at 2θ values of 19.6°, 21.6°, 31.7°, 32.6°, 35.3°, 38.9°, 41.5°, 43.3°, 46.1°, 53.7°, and 54.05°, corresponding to the (011), (−111), (−121), (121), (301), (022), (320), (400), (−213), and (132) crystal planes of monoclinic Ag₃VO₄ (JCPDS:43-0542), respectively.

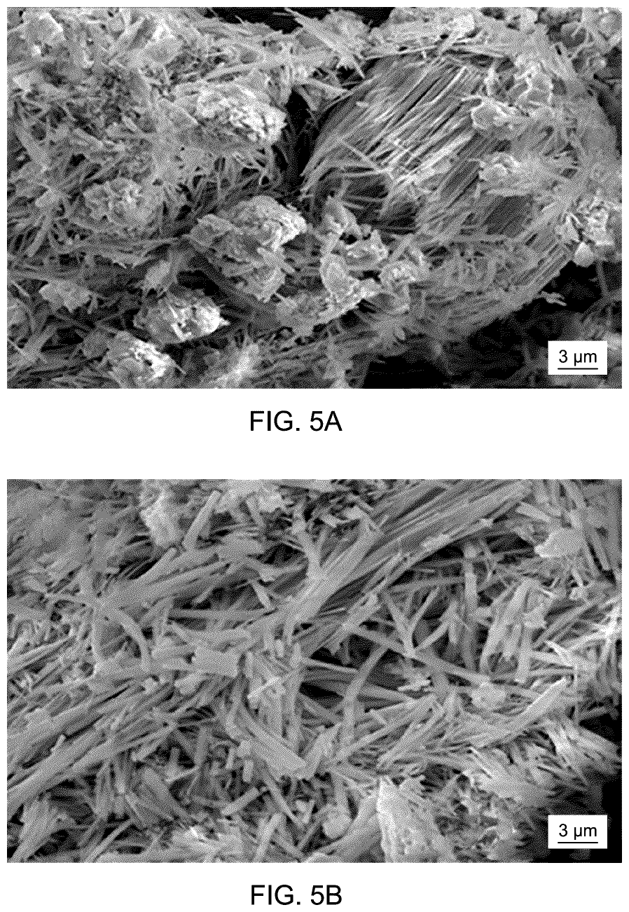

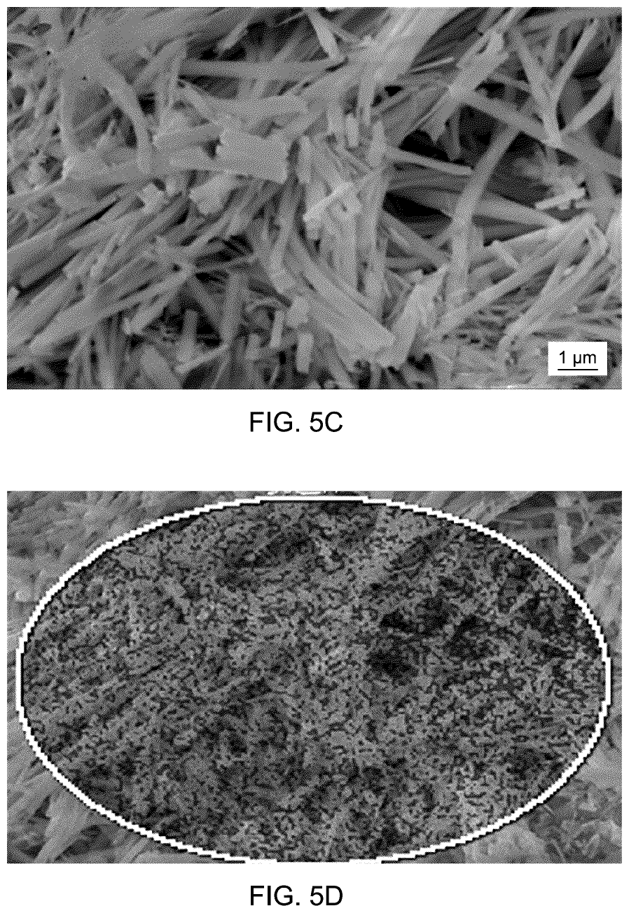

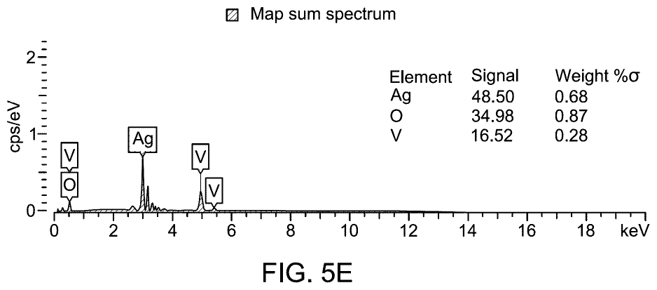

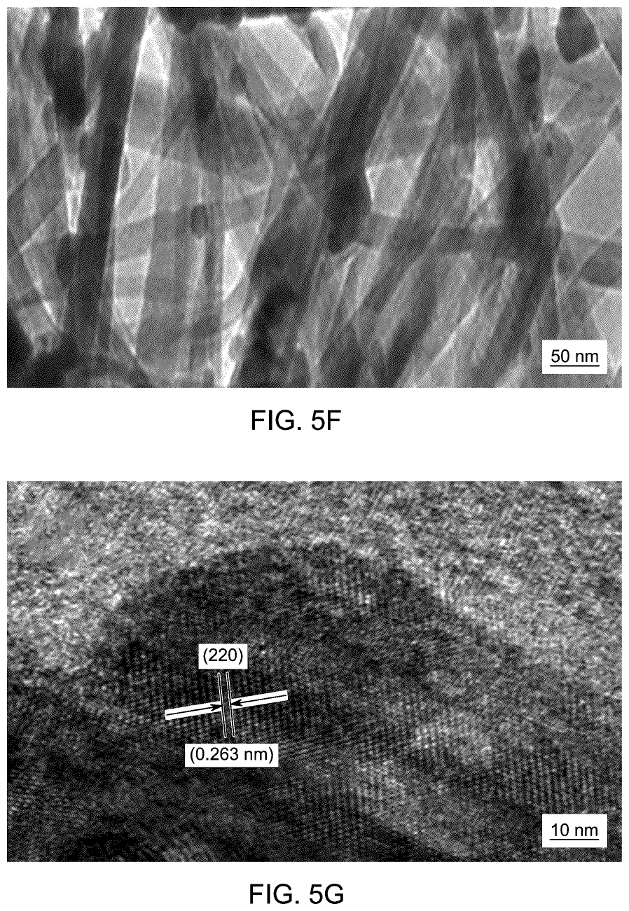

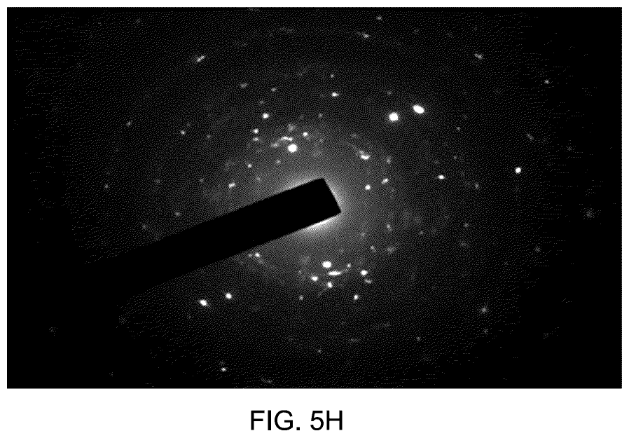

The investigation of Ag₃VO₄ nanorods involved the examination of morphology and microstructural features using field emission scanning electron microscopy (FESEM) and high-resolution transmission electron microscopy (HRTEM). FIGS. 5A-5H illustrate the FESEM and HRTEM images at various stirring stages. FIG. 5A and FIG. 5B reveal that the Ag₃VO₄ sample prepared after 6 and 12 hours of stirring, respectively, consisted of intermediate products including compact nanorods. However, with an increase in stirring time to 18 hours, a significant amount of nanorods formed, as displayed in FIG. 5C. The elemental composition, determined through energy dispersive X-ray spectroscopy (EDX) analysis in FIG. 5D and FIG. 5E, identified silver, vanadium, and oxygen. The TEM images of the pure Ag₃VO₄ photocatalyst, prepared after 18 hours of stirring, are presented in FIGS. 5F-5H. FIG. 5F displays the microstructure of Ag₃VO₄, composed of numerous compact smaller nanorods. The interplanar distance was measured at 0.263 nm, corresponding to the (220) plane of Ag₃VO₄, as presented in FIG. 5G. The selected area (Electron) diffraction (SAED) pattern in FIG. 5H exhibits a clear polycrystalline ring, indicating the good crystallization of Ag₃VO₄.

### Example 4. Electronic and Optical Characterization

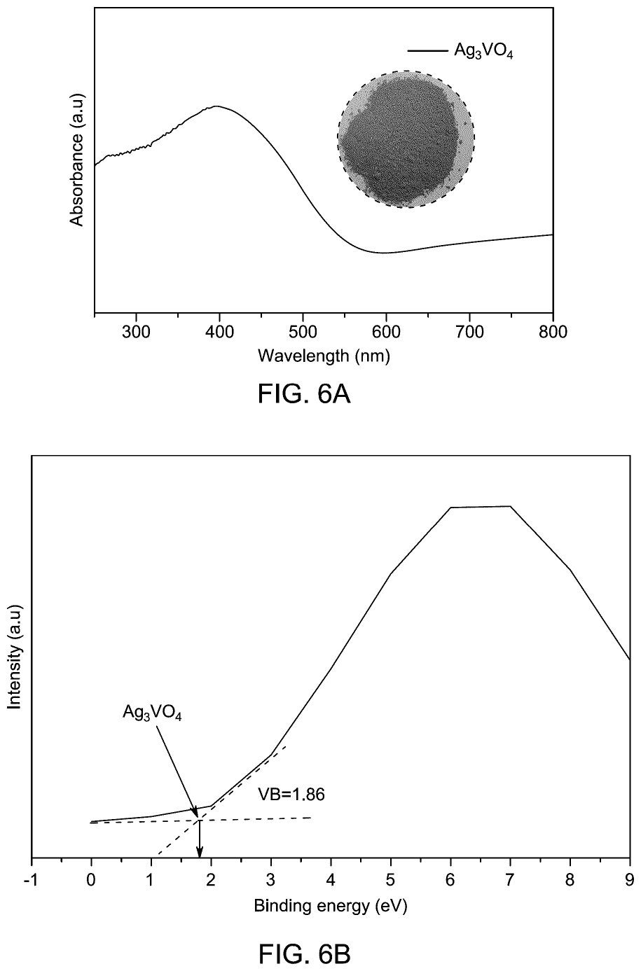

FIG. 6A illustrates the UV-visible diffuse reflectance absorbance spectra for the Ag₃VO₄ photocatalysts. It is evident that Ag₃VO₄ nanorods exhibit strong absorption intensities within the visible light range. The energy band gap (E_{bg}) of the Ag₃VO₄ photocatalyst was determined using the Tauc equation, as depicted in Equation (1).

$${E}_{g} ( eV ) = \frac{1240}{λ} \qquad ( 1 )$$

The Ag₃VO₄ photocatalyst exhibits a wavelength of 563 nm, corresponding to a calculated E_{g} value of 2.2 eV. The band gap determined from this analysis provides critical information about the electronic structure of the Ag₃VO₄ nanorods, including their capacity to absorb and utilize visible light. A smaller band gap generally indicates that the material can absorb a broader range of the visible spectrum, which is beneficial for photocatalytic applications, however a smaller band gap can also dramatically limit the available energy capable of being harnessed in a chemical transformation. Too small a band gap can effectively prevent certain desirable but energetically unfavourable reactions from occurring. Determining the band gap is essential for assessing the photocatalyst's efficiency and effectiveness in harnessing visible light for driving photocatalytic reactions, such as CO₂ reduction. The specific range of the band gap is a critical factor influencing the photocatalytic efficiency of the material, as it determines the extent of the photocatalyst's light absorption capabilities. The band gap value of 2.2 eV is particularly notable for its ability to effectively absorb visible light, which is essential for enhancing photocatalytic processes such as CO₂ reduction. This band gap value ensures that Ag₃VO₄ can utilize a significant portion of the visible light spectrum, thereby enhancing its photocatalytic performance and overall effectiveness in converting CO₂ into valuable solar fuels.

The conduction band position (E_{CB}) of the Ag₃VO₄ semiconductor was determined using Equation (2).

$${E}_{VB} = {E}_{CB} + {E}_{vg} \qquad ( 2 )$$

Valence band X-ray photoelectron spectroscopy (VB-XPS) was utilized to examine the valence band of Ag₃VO₄ photocatalyst. The conduction band and valence band are critical components of a semiconductor's electronic structure, dramatically affecting the photocatalytic activity. The conduction band represents the energy level where electrons can move freely and participate in chemical reactions, while the valence band is the lower energy band where electrons are typically bound to atoms. The energy gap between these bands, known as the band gap, determines the material's ability to absorb light and generate electron-hole pairs upon excitation.

Effective photocatalysts should ideally have well-aligned conduction and valence band edges to facilitate efficient charge separation and transfer, which are crucial for driving photocatalytic reactions, such as the reduction of CO₂ into solar fuels. The UV-visible diffuse reflectance absorption spectra of the Ag₃VO₄ photocatalysts reveal that the Ag₃VO₄ nanorods exhibit significant absorption within the visible light spectrum. Specifically, the spectra demonstrate a marked enhancement in absorption intensity across the visible range, which is indicative of the nanorods' effective light absorption capabilities. This pronounced absorption in the visible region may be advantageous for photocatalytic applications, as it signifies the Ag₃VO₄ nanorods' ability to efficiently utilize visible light for photocatalytic processes. The elevated absorption intensity indicates that these nanorods are well-suited for applications necessitating visible light activation, such as photocatalytic CO₂ reduction. For effective photocatalysis, it is essential that catalysts absorb light within the visible spectrum to drive the requisite photochemical reactions for converting CO₂ into valuable solar fuels. The strong absorption observed implies that the electronic band structure of the Ag₃VO₄ nanorods is favourably aligned with the energy levels required for photocatalytic activity. Consequently, this alignment enhances their capacity to generate electron-hole pairs upon visible light exposure, which is pivotal for efficient photocatalytic performance. The ability of the Ag₃VO₄ nanorods to absorb a substantial portion of the visible light spectrum may contribute to the overall efficacy and performance in photocatalytic applications.

Valence band X-ray photoelectron spectroscopy (VB-XPS) was utilized to examine the valence band of Ag₃VO₄ photocatalyst. As illustrated in FIG. 6B, valence band edges of Ag₃VO₄ were observed to be situated around 1.86 eV. The energy band gap (Ebg) for Ag₃VO₄ was determined to be 2.2 eV. Therefore, the conduction band of Ag₃VO₄ photocatalyst was calculated to be at −0.34 eV.

### Example 5: Photocatalytic Activity and Stability

The experiments examining the conversion of CO₂ with H₂O were conducted in the presence of visible light. During these tests, it was observed that when the lamp was turned off, there were no carbon containing compounds present in the reaction system. Conversely, under light exposure, a consistent generation of CH₄ and dimethyl ether (DME) was identified. This light provides the necessary energy to excite electrons from the valence band to the conduction band of the photocatalyst, thereby generating electron-hole pairs. The excited electrons in the conduction band become highly reactive and engage in the reduction of CO₂, leading to the formation of methane (CH₄), a valuable solar fuel. Simultaneously, the holes in the valence band facilitate the oxidation of water (H₂O), resulting in the production of oxygen (O₂) and hydrogen ions (H⁺). These hydrogen ions then participate in reactions with the CO₂, assisted by the excited electrons, to yield methane. The visible light acts as the driving force for these photochemical reactions, enabling the conversion of CO₂—a greenhouse gas into useful and energy-rich compounds.

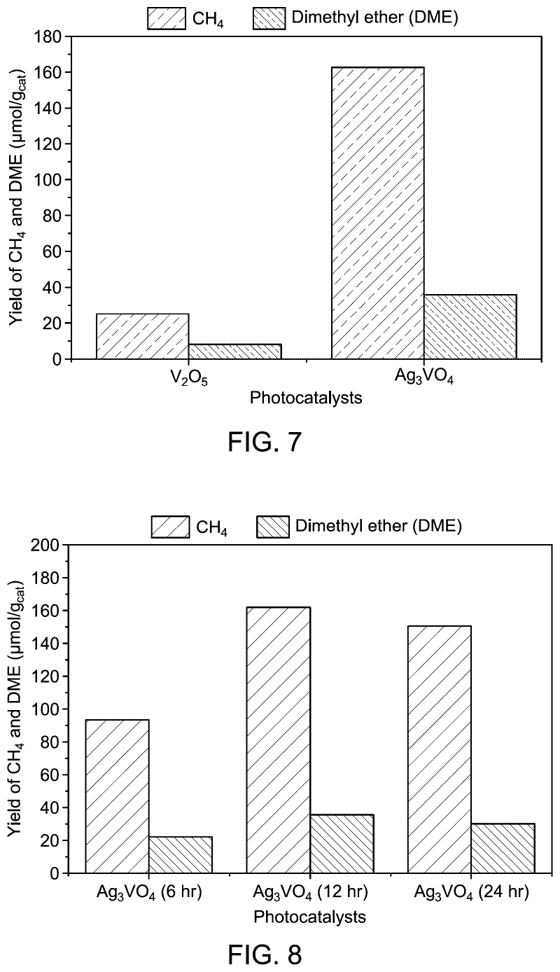

FIG. 7 illustrates a comparison of a V₂O₅ catalyst and the Ag₃VO₄ nanowire photocatalysts of the present disclosure on the photoactivity of photocatalytic CO₂ conversion under visible-light. The assessment of the photocatalysts focused on the yield of CH₄ and DME, the two resulting products. The pure V₂O₅ photocatalyst, synthesized through the sol gel process, demonstrated minimal CO₂ reduction and exhibited poor efficiency in generating CH₄ and DME, whereas the utilization of innovative Ag₃VO₄ nanorods significantly increased the production of CH₄ and DME. This improvement can be ascribed to the nanorods' effective absorption of visible-light, proficient charge transfer characteristics, and heightened electron mobility achieved through the coupling of vanadium with silver using the chemical deposition method.

FIG. 8 depicts the impact of varying photocatalyst preparation durations (6 b, 12 h, and 24 h) under room conditions and atmospheric pressure on the Ag₃VO₄ photocatalyst intended for CO₂ conversion with H₂O under visible-light. The efficiency of different stirring durations focused on the yield of CH₄ and DME, the two products resulting from the process. The findings reveal that the Ag₃VO₄ sample stirred for 24 h exhibits significantly enhanced photoactivity in the evolution of CH₄ and DME compared to samples stirred for 6 h and 12 h. Consequently, the Ag₃VO₄ sample prepared with a stirring duration of 24 h was chosen for further investigation into the effects of irradiation time and stability analysis.

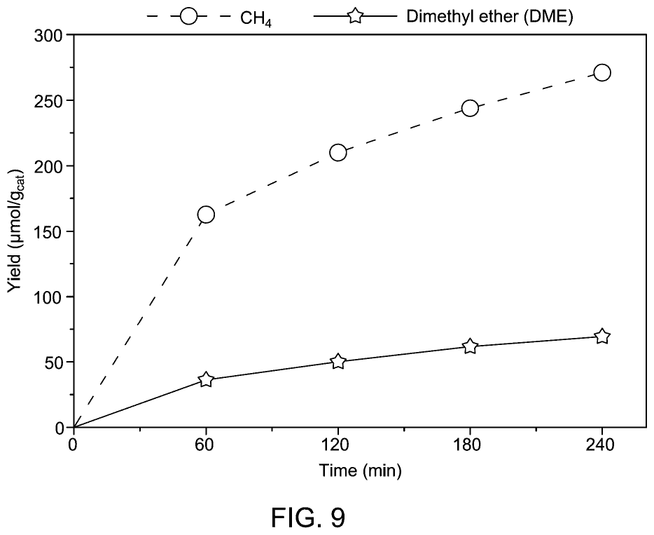

FIG. 9 plots the hydrocarbon production vs irradiation times on the visible-light driven photocatalytic conversion of CO₂ with H₂O to CH₄ and DME over Ag₃VO₄ nanorods. The gradual increase in CH₄ and DME generation becomes evident as the irradiation time extends. When employing Ag₃VO₄ nanorods, CH₄ emerges as the main product in the process of CO₂ photoreduction. Significantly, the catalyst exhibits sustained activity even after 4 hours of continuous irradiation, ensuring ongoing production of CH₄ and DME. Consequently, these innovative Ag₃VO₄ nanorods offer heightened photoactivity and stability, thereby enhancing the conversion of CO₂ into solar fuels.

### Example 6: Reaction Mechanism

Ag₃VO₄ nanorods serve as photocatalysts for assessing photocatalytic activity through the CO₂ conversion to CH₄ and DME. In the course of the reduction process, key reaction steps are succinctly outlined in Eqs. (3)-(7).

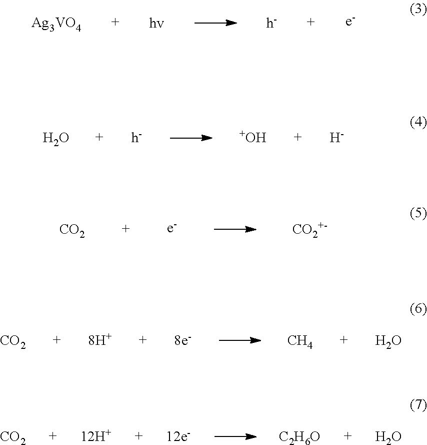

Eq. (3) illustrates the generation of electron-hole pairs upon photoexcitation. The conversion of CO₂ takes place in the conduction band through electron participation, whereas holes in the valence band facilitate the oxidation of H₂O, as elucidated in Eqs. (4) and (5). The mechanisms for producing CH₄ and DME via the reduction of CO₂ involving 6, 8, and 12 electrons are detailed in Eqs. (6)-(7). The investigation of photoactivity and reaction pathways provides valuable insights into the reaction mechanism.

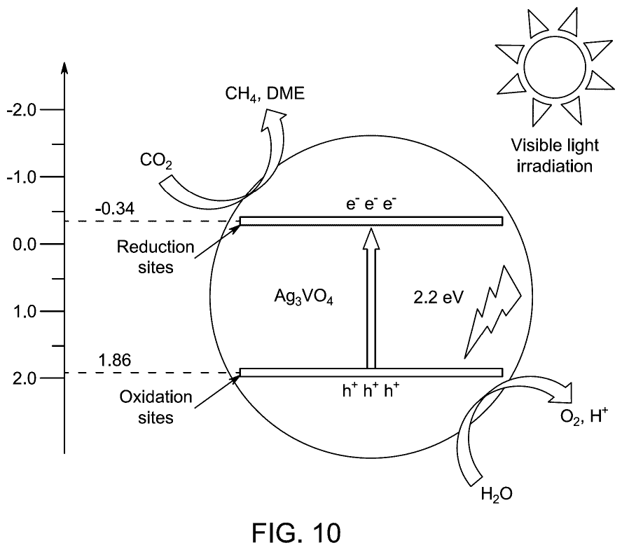

FIG. 10 illustrates a proposed reaction mechanism for the photo reduction of CO₂ to CH₄ and DME over Ag₃VO₄ nanorods under visible light. When exposed to visible light, electrons excited from the valence band (VB) of Ag₃VO₄ nanorods migrate to the conduction band (CB). Holes in the VB of Ag₃VO₄ nanorods interact with H₂O, leading to the generation of O₂ and H⁺. Concurrently, absorbed CO₂ molecules undergo reduction to form CH₄ and DME, facilitated by the enriched electrons on the surface of Ag₃VO₄. In the context of CO₂ reduction with H₂O, Ag₃VO₄ nanorods predominantly yield CH₄ as the main product, likely due to the suitable reduction potential of CO₂/CH₄ (−0.24 V). The reaction is more favorable for CH₄ production as the reduction potential of CO₂/CH₄ (−0.24 V) is lower than the conduction band of Ag₃VO₄. While C₂H₆O requires more electrons and has a conduction band closer to that of CH₄ compared to Ag₃VO₄ nanorods, the proper band alignment of Ag₃VO₄ contributes to the selective production of CH₄ during CO₂ conversion under visible light. Consequently, the Ag₃VO₄ nanorods exhibit significantly enhanced CH₄ production due to effective visible light absorption, a suitable band structure, and higher electron mobility with inhibited recombination.

Numerous modifications and variations of the present disclosure are possible in light of the above teachings. Therefore, it is to be understood that within the scope of the appended claims, the invention may be practiced other than as specifically described herein.

1. A photocatalyst, comprising catalytic Ag₃VO₄ nanowires, wherein:
the catalytic Ag₃VO₄ nanowires have a hierarchical structure comprising primary nanorods having a mean diameter of 2.5 to 50 nm arranged into nanowire bundles having a mean diameter of 100 to 500 nm.

2. The photocatalyst of claim 1, wherein
the catalytic Ag₃VO₄ nanowires are crystalline monoclinic Ag₃VO₄ by PXRD.

3. The photocatalyst of claim 1, wherein
the catalytic Ag₃VO₄ nanowires have a mean primary nanorod length of 1 to 15 μm.

4. The photocatalyst of claim 1, wherein
the photocatalyst is substantially free of V₂O₅, Ag₃O, and Ag₂O by PXRD.

5. The photocatalyst of claim 1, wherein the photocatalyst has
a band gap of 2.05 to 2.35 eV.

6. The photocatalyst of claim 1, wherein the photocatalyst has
a valence band energy of 1.71 to 2.01 eV; and
a conduction band energy of −0.64 to −0.04 eV.

7. The photocatalyst of claim 1, further comprising
a substrate on which the catalytic Ag₃VO₄ nanowires are disposed.

8. The photocatalyst of claim 7, wherein
the substrate is at least one selected from the group consisting of glass, quartz, indium tin oxide, fluorine tin oxide, and aluminum zinc oxide.

9. A method of forming the photocatalyst of claim 1, the method comprising:
mixing a first solution comprising a silver source in water with a second solution comprising a metavanadate salt in water to form a first reaction mixture;
aging the reaction mixture for 1 to 48 hours while stirring to form a first product;
washing the first reaction product with a distilled water to form a washed product; and
drying the washed product at 60 to 100° C. for 1 to 24 hours to form the catalytic Ag₃VO₄ nanowires.

10. The method of claim 9, wherein
the silver source is silver nitrate.

11. The method of claim 9, wherein
the metavanadate salt is ammonium metavanadate.

12. The method of claim 9, further comprising
disposing the catalytic Ag₃VO₄ nanowires on a substrate.

13. The method of claim 12, wherein
the substrate is at least one selected from the group consisting of glass, quartz, indium tin oxide, fluorine tin oxide, and aluminum zinc oxide.

14. A method of reducing carbon dioxide to methane, the method comprising:
contacting the photocatalyst of claim 1 with a gaseous mixture comprising carbon dioxide and water to form a reduction mixture,
irradiating the reduction mixture with visible light to form a product mixture comprising methane, and
collecting the product mixture.

15. The method of claim 14, wherein
the irradiating is performed with a visible light intensity of 50 W.

16. The method of claim 14, wherein
the gaseous mixture is contacted with the photocatalyst at a rate of 5 to 15 mL/min.

17. The method of claim 14, wherein
the method produces 120 to 200 μmol methane per g of photocatalyst after 1 hour of irradiation.

18. The method of claim 14, wherein
the gaseous mixture is substantially free of oxygen.

19. The method of claim 14, wherein
the product mixture further comprises dimethyl ether.

20. The method of claim 19, wherein
the product mixture has a ratio of methane to dimethyl ether of 1:1 to 8:1.
